(19) United States
(12) Patent Application Publication
(10) Pub. No.: US 2012/0270936 A1
(43) Pub. Date: Oct. 25, 2012

(54) COMPOSITION AND METHOD FOR TREATMENT OF INFLAMATION AND INFECTIONS OF THE GENITALIA, CONTRACEPTIVE AND THE PROPHYLAXIS OF SEXUALLY TRANSMITTED DISEASES

(76) Inventor: Joseph Di Bartolomeo, Santa Barbara, CA (US)

(21) Appl. No.: 13/442,735

(22) Filed: Apr. 9, 2012

Related U.S. Application Data
(63) Continuation of application No. 12/023,926, filed on Jan. 31, 2008, now abandoned.

(51) Int. Cl. A61K 31/375; A61P 15/00; A61M 31/00; A61P 31/04; A61P 29/00; A61F 6/04; A61P 15/02; A61P 31/18

(52) U.S. Cl. 514/474; 128/844; 604/286

(57) ABSTRACT

A composition, employed topically for its microbicidal, spermatocidal activity upon application to the human genitalia and/or anorectal area, and the relief from symptoms of local inflammation and infection incorporates L-Ascorbic acid in a concentration of about 2% to about 25% wt/vol, within an acidic range or pH level of about 4.0 to 2.0 pH respectively, in an aqueous solution with a pharmaceutically acceptable liquid carrier. In alternative exemplary includes a synergistic antioxidant and further incorporates enhancing bioactive ingredients. The method for topical application of the composition includes douche, rinse, cream, gel, suppository, saturated tampon and externally saturated condom for treatment of the vagina; drops, spray, cleansing wipe, cream, gel and internally saturated condom for treatment of the penis; and cleansing wipe, gel, suppository and externally saturated condom for male genitalia and rectum.

### REFERENCE TO RELATED APPLICATIONS

This application is a continuation of application Ser. No. 12/023,926 filed on Jan. 31, 2008 having the same title as the present application, the disclosure of which is incorporated herein by reference.

### BACKGROUND

1. Field of the Invention

This invention generally relates to treatment of inflammatory disorders due to microbes and biofilm products in the human body and more particularly to a composition and method which restores the natural pH balance to support the homeorrhesis in the human genitalia and anorectal area for synergistic cell division, tissue healing and autoimmune resistance against microbicidal activity while the local acidic milieu effects the degradation of the rheologic properties of the biofilm products of invading microbes and prevents their colonization, attachment, penetration and infection while further providing a contraceptive effect.

2. Description of the Related Art

In man, the mode of transmission of the more common diseases may be extrinsic or intrinsic. Extrinsic causes include the adherence by environmental microbes, toxins and pollutants to physical surfaces, and onto the host tissues. This may be in the form of airborne particles inhaled into the nose and sinus passages. Transmission frequently occurs by physical contact from handshakes, kissing or during sexual activities when the microbes are carried in the secretions or fluids of the body. The intrinsic causes are due to an alkalization of the normal acidic pH milieu of the host tissues and alteration of the composition of the protective surface mantel of the host organ and compromise of the immune defense mechanism of the host.

The mode of interaction is initially by surface contact, followed absorption of the surface nutrients, alteration of the normal commensal flora, colonization by microbes and release of their alkaline or basic pH biofilm and wastes.

The skin is the largest organ of the body. The surface epidermis is essentially a non permeable thick dry casing covering and protecting the internal organs of the body. The surface layer of keratin, a cornified scleroprotein, consists of dry sheets of millions of dead squamous epidermal cells that are completely shed approximately every thirty days. The epidermis is non-permiable and behaves as a lipid barrier. But, once hydrated, the skin becomes permeable and the bilayer barrier is penetrable.

At several regions of the body, the configuration of the skin conforms into pockets, passages or cavities which reach the internal organs of the body, and the substance of the tissue intergrades with additional complementary structures, glands and hair follicles, that maintain the organ homeorrhesis. Exemplary of such specialized organs are the human genitalia and anorectal area.

This transitional zone of changes in structure and function is seen in the vagina, penis and the anus of humans. These organs, without a keratinized (cornified) surface layer, instead have semi permeable membranes covered by vital secretions, both of which are completely regulated by the local milieu of osmolarity, acidity, pH, and nutrients for the host tissue homeorrhesis and immune defense system.

The lining of the genitalia are derivatives from skin primordium but the epithelium undergoes a dramatic and distinct transition within each organ. In the vagina, penis and the anogenital structures, the dry keratinous surface epidermis is replaced by a semi-permeable mucous membrane epithelium lining the passages. Within the entrance at the transitional zone, the sub-epithelial tissues contain a collection of hair follicles and specialized glands situated in an annular formation at the entrance, adapted to guard and protect the distinct host tissues.

The epithelium of each organ possesses specialized glands that secrete protective fluids that contain a microbicide enzyme to keep the unique commensal microbial flora in balance and to protect the host against colonization and infection by overgrowth of foreign surface microbes. The natural liquid product(s) maintain a specific level of acidity, or pH, to support the metabolic need of the local host tissues while being is inconsistent with the survival of microbes.

A disruption of this homeostasis will result in maceration, alkalosis of the liquid biofilm, tissue inflammation, infection and cellular disruption of the tissue defenses. Unless this natural acidic pH is restored, the microbes will adhere to the surface of the host, colonize within the protective biofilm barrier and then penetrate the outer cells of the host where they may infect the subsurface, target cells, unless they are stopped at the site of contact.

The external female genitals are the mons pubis, the clitoris, the labia majora, and the labia minora. Between the labia minora is the vestibule containing Bartholin's, Skene's and the urethral gland at the entry to the female copulatory organ, the vagina.

The vagina, the cervix, the uterus, the fallopian tubes, and the ovaries form the internal female genitalia. The vagina is a muscular, highly expandable, tubular cavity leading from the vestibule of the genitalia to the uterus. The cervix is the lower part of the uterus that protrudes into the vaginal canal. The uterus is a hollow, thick-walled, pear-shaped, muscular organ located between the bladder and rectum.

The adult female urethra is about 4 cm long and 8 mm in diameter. It is slightly curved and lies beneath the pubic symphysis just anterior to the vagina. The epithelial lining of the female urethra is squamous in its distal portion and pseudostratified or transitional in the remainder

The Epithelial cells lining the vagina are the first line of defense against pathogens. Epithelial cells are capable of synthesizing anti-microbial peptides that inactivate or recruit key immune cells. In addition, they stimulate the secretion of cytokines which support the survival of lymphocytes. Antibodies such as IgA and IgG are also abundant in the secretions in the vagina.

The vaginal desquamated tissue is made up of vaginal epithelial cells that are responsive to varying amounts of estrogen and progesterone. Superficial cells, the predominant cell type in women of reproductive age, predominate when estrogen stimulation is present. Intermediate cells predominate during the luteal phase because of progestogenic stimulation. Parabasal cells predominate in the absence of either hormone, a condition that may be found in postmenopausal women who are not receiving hormonal replacement therapy.

Normal vaginal secretions are composed of vulvar secretions from the Bartholin, sebaceous, sweat, and Skene's glands; a transudate from the vaginal wall; exfoliated vaginal and cervical cells; cervical mucus; endometrial and oviductal fluids; and microorganisms and their metabolic products. Bartholin's glands are two small, round structures, one on either side of the vaginal opening. These glands secrete a mucus-like fluid during sexual arousal, providing vaginal lubrication.

The type and amount of exfoliated cells, cervical mucus, and upper genital tract fluids are determined by biochemical processes that are influenced by hormone level. Vaginal secretions may increase in the middle of the menstrual cycle because of an increase in the amount of cervical mucus. These cyclic variations do not occur when oral contraceptives are used and ovulation does not occur.

The acidity of normal vagina secretions is usually at a low pH of about 4, maintained by the production of lactic acid by the Lactobacilli. The second source is the estrogen-stimulated vaginal epithelial cells which are rich in glycogen that is metabolized to monosaccharides which can then be converted to lactic acid by the cells themselves and by lactobacilli.

Unlike the human respiratory tract that has a mucociliary transport system to help remove any encroaching microbes, the vagina must rely on the critical mantel of Lactic acid produced by the dominant commensal microorganism—Lactobacillus, for defense on site.

The normal vaginal flora is predominantly aerobic, with an average of six different species of bacteria, including the Lactobacillus and Doderlein bacillus. The hydrogen peroxide-producing lactobacilli are the most common and cardinal bacteria: for the metabolizing of glycogen to secrete lactic acid in the vagina and maintains the critical low acid pH of the vagina, which provides a natural defense against proliferation of harmful microbes.

This low pH is achieved through the secretion of lactic acid by lactobacilli, the cardinal aerobic gram-positive rod that occurs naturally in the vagina and releases a variety of anti-microbial compounds such as lactic acid, hydrogen peroxide, bacteriocins, and biosurfactants.

The microbiology of the vagina is determined by factors that affect the survival of the bacteria flora. These factors include the vaginal pH and the availability of glucose to support the metabolism of these commensal bacteria. Human vaginal pH changes during the course of the menstrual cycle may fall to 4.2 at the time of ovulation. The naturally low pH of the vagina is affected substantially by the exogenous fluids of male semen which is alkaline and may substantially raise the pH level of the vagina resulting in the loss of this barrier to pathogens. Studies have demonstrated a variation of the pH of vaginal mucous when exposed to air for fertility studies.

Citric acid is one of the major chemical constituents of human semen secreted by the prostate gland with a distinctly alkaline pH.

These lactobacilli are sometimes destroyed by exogenous microbes that cause recurrent vaginal infections, e.g. bacterial vaginosis or sexually transmitted diseases and HIV.

Beneath the surface biofilm mantel of secretions of the vagina, and within the subepithelial tissues reside the immunodefensive Langerhan cells. These are dendritic star shaped cells in the stratum spinosum, the deeper portions of the germinative layer of the epidermis. They are rich in antigenic properties and class II major histocompatibility complex molecules.

These sub mucosal Langerhan cells are the primary target of the Human Imumodeficiency Virus (HIV). A disruption of the natural balance of the vaginal ecosystem enhances the risk of attachment and penetration by HIV.

A similar structure exists in the male penis and urethra. The penis is an external appendage composed of two corpora cavernosa and the corpus spongiosum, which contains the urethra, whose diameter is 8-9 mm. These corpora are capped distally by the glans. Each corpus is enclosed in a fascial sheath (tunica albuginea), and all are surrounded by a thick fibrous envelope known as Buck's fascia.

The skin covering the penis is devoid of fat, and is loosely applied about the fascia sheath casings. The prepuce, or foreskin, is that portion of the skin which forms a hood over the glans or head of the penis. The foreskin, like the vagina, is richly supplied with Langerhans and dendritic cells, but unlike the vagina, the epithelium of the inner foreskin is relatively thin and poorly keratinized at all times. By contrast, the glans penis has a highly keratinized epithelium to protect it from trauma during intercourse.

Although representing a relatively small segment of the digestive tract, the anal canal is anatomically unique, with a complex physiology that accounts for both its vital role in continence and its susceptibility to a variety of diseases. In the literature, two definitions are found to describe the anal canal. The “surgical” or “functional” anal canal extends for approximately 4 cm from the anal verge to the anorectal ring.

The lining of the anal canal consists of an upper mucosal and a lower cutaneous segment. The dentate (pectinate) line describes the “saw-toothed” junction of the ectoderm and the endoderm. It therefore represents an important landmark between two distinct origins of epithelial lining, the venous and lymphatic drainage and related nerve supply.

The cutaneous part of the anal canal consists of modified squamous epithelium-thin, smooth, pale, stretched, and devoid of hair and glands. The anal verge (anocutaneous line of Hilton) marks the lowermost edge of the anal canal and is sometimes the level of reference for measurements taken during colonoscopy or surgery. The stomal epithelium around the anus has acquired accessory structures; hair follicles, glands (including apocrine glands), and other features of normal skin.

Unlike its vaginal counterpart, the rectal epithelium provides little or no physical protection against potential trauma during intercourse, facilitating HIV-1 access to the underlying target cells, and even the systemic circulation. Moreover, the rectum, unlike the genital tract, is populated with organized lymphoid tissues (lymphoid follicles) that contain specialized microfold cells (m cells) that are capable of binding the presenting HIV-1 to the underlying lymphoid tissue. Such physiological and anatomical differences could account for the greatly increased risk of acquiring HIV-1 infection during anal intercourse. Indeed, intestinal epithelial cells can themselves transcytose HIV-1 particles to the underlying lamina propria when exposed to infected seminal leukocytes (macrophages or T cells). Although colorectal epithelial cells do not express CD4, they do express detectable levels of CXCR4, which, in theory, renders them susceptible to CD4-independent HIV-1 infection.

The Human Immunodeficiency Virus (HIV) is a disease that damages and destroys ones immune system, and ultimately causes death.

HIV interferes with the body's ability to effectively fight off viruses, bacteria and fungi that cause disease. This makes individuals more susceptible to certain types of cancers and to opportunistic infections your body would normally resist, such as pneumonia and meningitis. The virus and the infection itself are known as HIV. The term Acquired Immunodeficiency Syndrome (AIDS) is used to mean the later stages of an HIV infection, when the immune system is totally compromised and infection and death may follow.

In the 25 years since the first reports of the disease, AIDS has become a global epidemic. Worldwide, an estimated 42 million people are living with HIV, nearly half of them women and girls between the ages of 15 and 24. More than 25 million people have already died of AIDS. In 2005, more than 4 million people were newly infected with HIV. Heterosexual transmission of HIV is an important mechanism of transmission. Further, of the 4.8 million new infections and 2.9 million AIDS deaths in 2003, over 85% were in persons who acquired the HIV infection heterosexually. Not only is heterosexual transmission important in driving the current major epidemic in sub-Saharan Africa but is also a major factor driving the emerging epidemics in India and China.

In the countries worst affected by the HIV epidemic, women acquire HIV infection at a younger age, at least 5-10 years earlier than men. More than 1000 HIV-infected babies are born each day, often to teenaged mothers. Despite the effectiveness and availability of the condom, the HIV epidemic continues to spread. There are an estimated 5 million new HIV infections per year, with more women than men now becoming infected. Sexual transmission is mediated by exposure to infectious HIV-1 and/or infected cells in the semen or mucosal secretions. The relative transmissibility of cell-free virions versus a cell-associated virus is still uncertain, but both sources of the virus should be targeted by intervention strategies.

The risks of transmitting or acquiring infection vary greatly. Epidemiological studies strongly indicate that transmission is linked to viral shedding, that is, the amount of infectious virus that is present in genital fluids. This is in turn linked to the disease stage, and is highest during acute infection and late-stage AIDS. Effective antiviral therapy can reduce HIV-1 shedding in semen and the female genital tract to undetectable levels, but HIV-1 can sometimes be found in semen even when undetectable in the bloodstream.

Therefore, although some infected individuals pose little transmission risk, others could be ‘super-shedders’ and highly infectious throughout (or intermittently during) the course of infection. Acutely infected individuals pose a particularly profound risk, which is why epidemics usually spread explosively when they strike a new population; highly viremic people unaware of their newly infected status are at high risk for transmitting the infection to new individuals who then become highly viremic while remaining sexually active—a vicious spiral. Moreover, other sexually transmitted diseases (STDs) have a marked effect on both viral shedding and increase the risk of acquiring the HIV-1 infection. The most sexually active population is also the one that is most at risk for STDs, an the establishment of one infection that compromises the host immune system, increases the risk of secondary HIV-1 infections among young, sexually active adults.

### HIV Treatment Options

Generally, the action of Microbicides can be classified as nonspecific, moderately specific or highly (exclusively) specific to microorganisms, host or both. The nonspecific and moderately specific agents are often active against a variety of sexually transmitted microorganisms (e.g., chlamydia and herpes virus) and/or sperm with a contraceptive effect, or may impact the host tissues negatively. The HIV-specific agents interact directly with one or several steps of the infection or replication cycle of the HIV.

### Current Systemic Treatment of Established Infections

Systemic microbicides have been developed to maintain the colonization of the vagina by lactobacilli or to recolonize the vagina with lactobacilli when these commensal organisms have been adversely affected by the use of antibiotics for genital tract infections. The mechanism of action of the systemic microbicides is to interrupt the route of entry of HIV in the target cells of the female reproductive tract at the specific HIV receptors, namely the Langerhans cells and other dendritic cells within the vaginal epithelium.

Tenofovir (trade name Viread) is an anti-HIV drug approved by the FDA to be used in combination with other HIV fighting medications. Viread belongs to a new class of drugs called nucleotide reverse transcriptase inhibitors (NtRTI). These are related to nucleoside reverse transcriptase inhibitors (NRTI) like zidovudine (AZT, Retrovir). The body converts Viread into a chemical that prevents HIV from reproducing in uninfected cells, but it does not help cells that have already been infected with the virus. As people with HIV lose CD4 cells—one of the immune system's main defenses—they become more likely to get infections and illnesses.

In the clinical trials, volunteers were having difficulty suppressing HIV, about 94% of the volunteers had at least one NRTI (like AZT) resistant strain of HIV.

The known potential side effects of Viread and other microbicides are nausea, diarrhea, vomiting and flatulence. A set of rare but serious side effects of nucleoside analog anti-HIV drugs is called lactic acidosis and severe hepatomegaly with steatosis (an enlarged fatty liver) and kidney problems.

The observed level of toxicity observed in these microbicides suggests that pathological effects may become apparent if these compounds are needed at higher concentrations or for longer durations.

The problem with developing more complex systemic antiretroviral drugs is that the virus can mutate far faster than the pharmaceutical companies can develop such therapies.

Despite improved treatments and better access to care for people in the hardest-hit parts of the world, most experts agree that the pandemic is still in the early stages. With a vaccine probably decades away, and constant viral mutations making treatment ineffective, the best hope for stemming the spread of HIV now lies in prevention.

### Prevention

Topical microbicides have been produced as a possible new therapeutic approach to stop the HIV upon initial contact. These microbicides have been developed specifically on the principle of assisting the vagina in maintaining a low pH even in the presence of alkaline semen. They are formulated as gels, foams, films or vaginal rings designed to be inserted into the vagina or rectum and meet the urgent need for an effective female-controlled method of HIV prevention. More than 60 potential microbicides are being assessed in preclinical and clinical trials.

The “first generation” topical microbicide candidates were surfactants administered for their effect on epithelial adherence. These products had detergent-like properties to disrupt cell membranes or, in some instances, changed the cell's membrane structure to make it more porous and thereby more liable to disruption. But these products impacted on all living cells: the host, commensal and pathogenic organisms. These products exhibit a wide spectrum of indiscriminate activity against several living cells; including most microbes but destroy human spermatozoa and even the host tissues, as well.

A well published example was topical nonoxynol 9, which was widely used throughout the world as a microbicide until studies in African commercial sex workers showed that frequent use damaged the vaginal epithelium and increased their susceptibility to the HIV infection (Van Damme 35 al. 2002).

A disruption of the natural balance of the vaginal ecosystem enhances the risk of the HIV. Although HIV appears to remain stable in a slightly acidic environment, there is a substantial reduction in infectivity when the pH levels are reduced below 4.5. Further lowering of the acidic pH to the level of the method described herein has demonstrated that the HIV is inactivated.

As outlined above, several factors account for the explosive spread of any newly arrived HIV strain in an ‘at-risk’ population. Every genetic subtype is transmissible through vaginal or rectal intercourse; none should be ignored or considered to be less of a problem.

### Mechanisms of Sexual HIV-1 Transmission

Mucosal physiology provides a significant initial hurdle for HIV-1 transmission. The multiple layers of stratified squamous epithelium that line the most exposed regions of the female and male genital mucosa (vagina and ectocervix in women; inner foreskin, penile glans and fossa navicularis in men) constitute a significant physical barrier to an incoming virus. The epithelium in these regions has limited permeability to particles that have a diameter greater than 30 nm; the diameter of an HIV-1 virion is 80-100 nm.

For sexual transmission to occur, infectious HIV-1 must cross the mucosal epithelium. Several mechanisms for sexual transmission have been proposed, however, whether any, or all, of these mechanisms fully explain HIV-1 transmission in vivo is uncertain. Moreover, infection at different tissue sites might involve different mechanisms. Numerous intercellular desmosomes and abundant amorphous lipidic material are crucial components of the integrity of the epithelial barrier. As cells progress outwards from the basal layers, they become flattened and keratinized, although they are not cornified. Consequently, the outermost, apical surface of the genital epithelial barrier comprises a superficial layer of dead epithelial cells that is impervious to the virus and is renewed every three days. The effectiveness of this barrier is shown by the dramatic increase in the susceptibility of rhesus macaques to vaginal simian immunodeficiency virus (SIV) transmission when the epithelial layer is thinned by progesterone treatment.

In line with their protective function, stratified genital epithelial cells are not susceptible to HIV-1 infection and do not transcytose viral particle. However, these cells might be able to bind viral particles on their surface and thereby facilitate the infection of other cell types in trans. Furthermore, studies in both animal models and human tissue ex vivo provide little evidence that donor cells can transmigrate across such epithelia. Therefore, infected cells are more likely to provide a localized reservoir of virus that is external to the epithelium itself.

Some degree of breakdown in epithelial integrity might be required for HIV-1 transmission in vivo, and this occurs frequently. Epithelial micro-abrasions can be detected in 60% of women following consensual intercourse, and are also frequently observed on the inner foreskin and penile glans. Indeed the large surface area of the foreskin increases the potential for microtrauma in uncircumcised men, perhaps explaining the reduced risk of infection that is associated with circumcision. The protective effects of the stratified epithelium are further diminished by the ulcerative STDs that increase the risk of transmission. Overall, anything that impairs epithelial integrity, such as, physical abrasion or trauma, ulceration, inflammation, hormonal imbalance (increased progesterone), low micronutrient levels or the use of intravaginal preparations for “dry sex”, can critically increase susceptibility to HIV-1 infection.

### Potential Target Cells for HIV-1 Infection

Genital and rectal subepithelial stromal tissues are densely populated with DCs, macrophages and T cells that express CD4, CCR5 and, to a lesser extent, CXCR4. Each of these cell types is therefore susceptible to HIV-1 infection. Any extensive breakdown in epithelial integrity allows HIV-1 direct access to a rich source of target cells, allowing the establishment of infection in mucosal sites. Indeed, several studies using macaque transmission models and human mucosal tissues ex vivo have identified subepithelial T cells, macrophages and DCs as the primary targets for infection. Infection of these cells can be detected within 1 hour of the addition of SIV to the macaque vagina, and is most commonly observed where the epithelium is abraded, further emphasizing the protective nature of an intact epithelium.

The role coreceptors play in HIV binding and of the particular role dendritic cells play in transmitting the virus to the lymphatic system is cardinal to treatment.

the viral envelope has an affinity for binding to the CD4 protein on the host cell, and a host T-cell is needed for HIV to enter the cell. Dendritic cells are immune cells with thread-like tentacles or dendrites that “capture” antigens and transport them to T cells. Examples of dendritic cells include Langerhans cells found in the skin and mucosal membrane, and follicular dendritic cells found in lymphoid tissue. The bound HIV migrates to lymphatic areas, where T4 lymphocytes can be productively infected.

If the HIV virus can penetrate the epithelial barrier of the vagina, an infection may develop in the Langerhans cells. Once the HIV fuses with the target host cells, any infection that follows that is extremely difficult to eradicate.

New preventive therapeutic strategies are urgently needed that are void of these serious side effects. Prevention is preferred to treatment which is cost prohibitive in most clinical settings and carries a risk of serious side effects.

### Female Infections

It is important to recognize that women are more vulnerable to diseases of the genital tract than men. The lining of the vagina is a mucous membrane and more permeable than the outside of the penis, and women a greater internal surface area through which infection can occur. Lack of lubrication during intercourse, changes in the cervix during the menstrual cycle, and asymptomatic infections facilitate more efficient transmission of infection to women. Prepubertal girls and adolescents are particularly vulnerable, because their vaginal and cervical tissues may be less mature and more readily penetrated by organisms (e.g., chlamydia and gonococcus). Postmenopausal women are more likely than younger women to get small abrasions in the vagina during sexual activity as a result of thinning of the tissue and dryness. Other biological risks include the use of vaginal douches, which increase the risk of pelvic inflammatory disease, and the influence of hormonal contraceptives on acquiring or transmitting an STD (e.g., increased risk of chlamydial infection with use of oral contraceptives), though this is not fully understood.

It is been postulated that repeated alkalization of the vagina, which occurs with high basic pH level of semen in frequent sexual intercourse or the use of douches, plays a role. As normal hydrogen peroxide-producing lactobacilli disappear, the defense is weakened, other bacteria present in the vagina proliferate (e.g. Bacteroides sp, Peptostreptococcus sp, Garnerella vaginalis, G. mobiluncus, Mycoplasma hominis) and as the vaginal pH increases, symptoms occur. BV is associated with a history of STDs, frequent douching, overused or retained tampons, intrauterine contraceptive devices (IUDs), diaphragms, contraceptive sponges, use of broad spectrum antibiotics and products containing nonlxynol-9.

Women who already have an infection (particularly one that causes genital lesions) are more likely to get or transmit another sexually transmitted disease (STD), including the HIV.

Chlamydia: More than 1 million cases of chlamydia were reported in the United States last year—the most ever reported for a sexually transmitted disease, federal health officials said Tuesday. “A new U.S. record,” said Dr. John M. Douglas Jr. of the Centers for Disease Control and Prevention. More bad news: Gonorrhea rates are jumping again and an increasing number of cases are caused by a “superbug” version resistant to common antibiotics, Federal Officials stated. Syphilis is rising, too. The rate of congenital syphilis—which can deform or kill babies—rose for the first time in 15 years. The CDC releases a report each year on chlamydia, gonorrhea and syphilis, three diseases caused by sexually transmitted bacteria. But Chlamydia is the most common. Nearly 1,031,000 cases were reported last year, up from 976,000 the year before.

Bacterial Vaginosis (BV) is the most common form of vaginitis in the United States. BV has previously been referred to as nonspecific vaginitis or Gardnella vaginitis. It is an alteration of the natural balance of the commensal aerobic bacterial flora in the vagina. Bacterial vaginosis accounts for 60% of vulvovaginal infections. BV is caused by a change in local pH and the natural balance of bacteria in the vagina. It is not known what triggers the disturbance of normal vaginal flora, but young adult women, particularly those who are sexually active, are most commonly affected.

BV results from a loss of hydrogen peroxide-producing lactobacilli and an overgrowth of predominantly anaerobic bacteria (3,4). Anaerobic bacteria can be found in less than 1% of the flora of normal women. In women with BV, however, the concentration of anaerobes, as well as G. vaginalis and Mycoplasma hominis, is 100 to 1000 times higher than in normal women. Lactobacilli are usually absent.

BV is diagnosed on the character of the secretions and the development of a distinct odor. The pH of these secretions is higher than 4.5 (usually 4.7-5.7). After the protective hydrogen peroxide-producing lactobacilli disappear, it is difficult to reestablish abnormal vaginal flora, and recurrence of BV is common.

Trichomonal Vaginitis is caused by the sexually transmitted, flagellated parasite, T. vaginalis. The parasite is anaerobic, and has the ability to generate hydrogen to bind with oxygen to create such an anaerobic environment. As a result, The pH of these vaginal secretions is usually higher than 5.0. The transmission rate is high.

Vulvo Vaginal Candidiasis (VVC). It is estimated that as many as 75% of women experience at least one episode of (VVC) during their lifetimes. Almost 45% will experience two or more episodes.

Candida Albicans is responsible for 85-90% of vaginal yeast infections. Factors that predispose women to the development of symptomatic VVC include antibiotic use, pregnancy, and diabetes. Antibiotic use disturbs the normal vaginal flora and eliminates the protective lactobacilli along with the other normal flora. This change alters the pH to favor the perpetuation of the VVC, until the normal flora can be restored.

Studies by Hilmarsson demonstrated the virucidal activity profiles of fatty alcohols, lipids at a low pH. His finding that fatty alcohols and lipids are more active against VV at pH 4.2 than at pH 7 is in agreement with earlier studies, which have shown that lipids become generally more virucidal at low pH. The increased virucidal activity against enveloped viruses at low pH may be due to ionic changes in the glycoproteins on the surface of the viral envelope, thus giving the lipid molecules better access to the lipid bilayer of the envelope in acidic environment than at neutral pH.

A pH of 4 is also known to kill other STDs in culture, such as Chlamydia, HSV-2, Neisseria gonorrhoeae, Treponema pallidum and HIV-2 while leaving the commensal Lactobacillus acidophilus unharmed. It has long been known that dilute lemon juice is a very effective topical spermicide, because of the low pH produced by its citric acid content (Himes 1963).

Tested in a culture medium, citric acid had little buffering capacity, so a 20% concentration of lime or lemon juice reduced the pH to 2.9, exhibiting spermicidal activity and inactivating 90% of the HIV in 2 min. while 10% concentration (pH 3.4-3.7) inactivated up to 50% in 2 min. But unfortunately, at the former concentrations, citric acid acts as a corrosive agent, destroying all tissues, including the defensive vaginal lining.

What is needed is a microbicide that preserves the pH of the acidic mantel for normal primary vaginal defenses, with spermicidal effects and at the initial point of contact, and with antioxidant co-enzymes factors that support the production of pre-collagen for host tissue strength. But it must be universally safe, both for teenagers and the sexually age group, and the delicate host tissues.

### Male Infections

It appears that HIV enters the human penis through the inner aspect of the foreskin, which is richly supplied with both Langerhan and dendritic cells, Prophylaxis, the protective effect of male circumcision would eliminate one point of entry of the HIV.

### Male Post-Coital Hygiene

How long following first contact with HIV does it take for a man's penis to become infected? The virus must become attached to specific HIV receptors in the penile epithelia, and studies in Rhesus monkeys in which SIV has been applied to the foreskin have suggested that it may take an hour or more before the virus is internalized (Miller 1998). Thus post-coital penile hygiene, wiping the penis, and in particular the inner aspect of the foreskin, with a potent microbicide within minutes of penile withdrawal from the vagina (or rectum) may significantly reduce a man's chances of becoming infected (Short, 2004).

### Rectum

In the past, antibiotics have been the mainstay of eliminating bacterial microorganisms by direct biochemical interaction. Antibiotics can only treat the infection when and if the agent is capable of penetrating the biofilm barrier and altering the biochemistry of the microorganism internally. This is seldom the case now with mutations resulting in more resistant biofilm surface shields.

With the past overuse of antibiotics and the development of resistance by microorganisms now to newer antimicrobial treatment, such therapy has become less effective and frequent serious side effects have become common.

More recently, because the complex chemical structures of the newer anti-infective agents are not absorbable in the gastrointestinal tract, the medication must be administered by intravenous injection. This mode is associated with serious side effects such as ototoxicity, hearing loss and tinnitus, allergic reactions, anaphylaxis and death.

It is therefore desirable to provide a novel method and composition for the prevention and treatment of symptoms, and inflammatory disorders due to microbes and biofilm products of the vulnerable specialized target organs including the vagina, penis, anus and rectum of humans.

It is also desirable if the method and composition additional provides a contraceptive effect.

Materials relied upon with respect to the information provided in the Background of the application are defined in the following Bibliography for proper attribution.

### BIBLIOGRAPHY

Abdool, Salim S. MBChB, PhD., Microbicides for the Prevention of HIV Infection.

Balzarini, Jan and Van Damme, Lut, Intravaginal and intrarectal microbicides to prevent HIV infection, CMAJ, Feb. 15, 2005, 172 (4).

Barnhart, Kurt, Izquierdo, A., Pretorius, E S, Shera, D M, Shabbout, M and Shaunik, A. Baseline dimensions of the human vagina, Human Reproduction, Vol. 21, No. 6 pp. 1618-1622, 2006.

Barnhart K., Pretorius E., Timbers K, Shera D., Shabbout M., Malamud D., Distribution of a 3.5-mL (1.0%) C31G vaginal gel using magnetic resonance imaging. Contraception, Volume 71, Issue 5, Pages 357-361.

Berek, Jonathan (Editor), Novak's Gynecology, Thirteenth Edition, Lippincott Williams & Wilkins.

Bhoopat L, Eiangleng L., Rugpao S., Frankel S., Weissman D, Lekawanvijit S., Petchjom S., Thorner and Bhoopat T., In Vivo Identification of Langerhans and Related Dendritic Cells Infected with HIV-1 Subtype E in Vaginal Mucosa of Asymptomatic Patients, Mod Pathol 2001;14(12):1263-1269.

Bowie, Andrew G and O'Neill, Luke A. J., Vitamin C Inhibits NF-kB Activation by TNF Via the Activation of p38 Mitogen-activated Protein Kinase, Journal of Immunology, 2000, 165:7180-7188.

Burns, J. J., Missing Step in Man, Monkey and Guinea Pig required for the Biosynthesis of L-Ascorbic Acid. Nature, Letter to the Editor, No. 4585, Sep. 14, 1957: 553.

Carballo-Dieguez, Alec, Stein, Zena, Saez, Helga, et al., “Frequent Use of Lubricants for Anal Sex Among Men Who Have Sex with Men: The HIV Prevention Potential of a Microbicidal Gel, American Journal of Public Health, July 2000, Vol. 90, No. 7, Pg 1117-1121.

Correa, C H M, Mattos, A L G and Ferrari, A N, In situ variation of cervical mucous pH during exposure to atmospheric air, Braz J Med Bio Research (2001) 34: 767-770.

Dunne, Jr., W. M. FOCUS, Bacterial Adhesion: Seen Any Good Biofilms Lately? Clinical Microbiology Reviews, April 2002, 155-166.

Engler, M., Engler, M., Malloy, M., Chiu, E., Sclotoetter, M., Paul, S., Stuehlinger, M., Lin, K., Cooke, J., Morrow, J., Ridker, P., Rifai, N., Miller, E., Witzum, J., Mietus-Snyder, M. Antioxidant vitamins C and E improve endothelial function in children with hyperlipidemia. Circulation 2003: 108; 1059-1063.

Gallarate, M., Carlotti, M., Trotta, M., Bovo, S. On the stability of ascorbic acid in emulsified systems for topical and cosmetic use. International Journal of Pharmaceutics 1999; 188: 233-241.

Hilmarsson Hilmar, Larusson Lazarus, Halldor T. Virucidal effect of lipids on visna virus, a lentivirus related to HIV, Archives of Virology, Vol 151, No 6, June 2006, 1217-1224. (Institute of Biology, University of Iceland. hilmarh@hi.is).

J. J. Homer, R. J. England, A. D. Wilde, G. R. J. Harwood & N. D. Stafford, The effect of pH of douching solutions on mucociliary clearance, (1999) Clin. Otolaryngol. 24, 312-315.

Lee, J., Kim, J., Han, S., Chang, S., Kang, H., Lee, O., Oh, S., Suh, K. The stabilization of L-ascorbic acid in aqueous solution and water-in-oil-in-water double emulsion by controlling pH and electrolyte concentration. J. Cosmet. Sci January/February 2004; 55: 1-12.

Lianou, P. E., Fortis, A. A., Papavassilliou, J. T., The effect of low concentrations of ascorbic acid in microbial adherence in vitro. Int J Vitam Nutr Res. 2003 July; 73 (4): 245-50.

The Maillard Reaction: Chemistry at the Interface of Nutrition, Aging, and Disease Edited by: John W. Baynes, University of South Carolina, Charleston, S.C.; Vincent M. Monnier, Case Western Reserve University, Cleveland, Ohio; Jennifer M. Ames, University of Reading, Whiteknights, Reading, UK; Suzanne R. Thorpe, University of South Carolina, Columbia, S.C.

Mann, T. 1964 The biochemistry of semen and of the male reproductive tract. London: Methuen.

Margolis, Sam A., and Park, Edward, Stability of Ascorbic Acid in Solutions Stored in Autosampler Vials, Clinical Chemistry 47, No. 8, 2001, pp 1463-1464. (NIST, Analytical Chemistry Division, Gaithersburg, Md. 20899-8392, author for correspondence: fax 301-977-0685, e-mail sam.margolis@nist.gov).

McCoombe, Scott G., Short, Roger V., Potential HIV-1 target cells in the human penis, AIDS 2006, 20:1491-1495.

O'Connor T J, Kinchington D, Kangro H O and Jeffries D J., Second International Congress, Drug Therapy in HIV Infection of Low Ph Genital Secretions and Virucidal Agents Against HIV-1, Int Cong Drug Therapy HIV 18-22:2:Abstract No. P127, AIDS 1994, Vol. 8 (Suppl. 4);S50.

Pendergrass Paula, Belovicz Meyer, Reeves Cornelia, Surface Area of the Human Vagina as Measured from Vinyl Polysiloxane Casts, USA Gynecologic and Obstetric Investigation 2003;55:110-113 (Department of Biological Sciences, Arkansas Tech University, Russellville, Ark.)

Petersen, E., Magnani, P., Efficacy and safety of Vitamin C vaginal tablets in treatment of non-specific vaginitis, European Journal of Obstetrics—Gynecology Reproductive Biology, 2004, Vol. 117, pg. 70-75.

PUBLIC HEALTH HIV Infogram, “Update on Sexual Transmission of HIV, March 2002—HIV/AIDS Program”, Public Health, Seattle & King County.

Rawal, B. D. Bactericidal action of ascorbic acid on Pseudomonas aeruginosa-alteration of cell surface as possible mechanism. Chemotherapy (1974), 24, 166-172.

Shattock R J, Moore J P., Inhibiting sexual transmission of HIV-1 infection. Nature Rev Microbiol 2003;1:25-34.

Short, R. V., New ways of preventing HIV infection: thinking simply, simply thinking, Phil. Trans R. Soc. B (2006) 3621, 811-820.

Short, R. V., McCoombe, S, Maslin, C, Naim, E, Crowe, S, Lemon and Lime juice as potent natural microbicides. Dept of Obstetrics & Gyn, The University of Melbourne, Australia.

Tanagho, Emil and McAnich, Jack W (Editors) Smith's General Urology, Fifteenth Edition, Lange Medical Books/McGraw-Hill.

Van Robertson, W. The effect of ascorbic acid deficiency on the collagen concentration of newly induced fibrous tissue. From the Division of Experimental Medicine and the Department of Biochemistry, College of Medicine, University of Vermont, Burlington, Vermont, Journal of Biological Chemistry, 1952 May; 196(1): 403-8.

Van Robertson, W., Schwartz, B. Ascorbic acid and the formation of collagen. From the Division of Experimental Medicine and the Department of Biochemistry, College of Medicine, University of Vermont, Burlington, Journal of Biological Chemistry, 1953 April; 201(2): 689-696.

Wagner, G. & Ottesen, B. Vaginal physiology during menstruation. Ann Intern. Med. 1982, 96, 921-923.

Zhu T, Wang N, Carr A, Nam D S, Moor-Jankowski R, Cooper D A, Ho D D, “Genetic characterization of human immunodeficiency virus type 1 in blood and genital secretions: evidence for viral compartmentalization and selection during sexual transmission” J. Virol., May, 1996, 3098-3107, Vol. 70, No 5.

### SUMMARY OF THE INVENTION

The present invention provides a composition for topical application to the human genitalia and anorectal area for relief from symptoms of inflamation and microbial infections, the prophylaxis of sexually transmitted diseases (STD's) and contraception. The embodiment incoporates L-Ascorbic acid in a concentration of about 2% to about 25% wt/vol., within an acidic range or pH level of about 4.0 to 2.0 pH respectively, in an aqueous solution with a pharmaceutically acceptable liquid carrier. The liquid carrier in alternative exemplary embodiments is pure distilled water or normal 0.9% physiologic saline, pH adjusted in a predetermined composition for the desired therapeutic effect. Additionally, the composition in an alternative embodiment includes a synergistic antioxidant such as Tocopherol, water solubilized vitamin E (alpha-tocopherol) in a concentration of about 500 to 1,000 i.u./100 m or a flavone, bioflavonoid, in a concentration of about 0.1 to 0.5% wt/vol. Additionally, the composition further incorporates zinc gluconate in certain embodiments.

The method for topical application of the composition includes douche, rinse, cream, time-released gel, suppository, saturated tampon and externally saturated condom for treatment of the vagina; drops, spray, cleansing wipe, cream, gel and internally saturated condom for treatment of the penis; and cleansing wipe, gel, suppository and externally saturated condom for treatment of the rectum.

### DETAILED DESCRIPTION OF THE INVENTION

The isotonicity, osmolarity together with the acidity (the pH level), of extracellular and intracellular fluids, govern the vital biochemical and metabolic processes of all living tissues, including microbes. The relative acidity of a solution or tissue is measured according to a pH scale that ranges from 0-14. Those registering above 7 function within an alkaline environment, those below 7 are acidic. A solution with a pH of 7 is considered neutral.

The mathematical symbol pH represents the logarithm of the reciprocal of the hydrogen-ion concentration in gram atoms per liter of solution. The level of the pH regulates the vital biochemical availability of nutrients for the immune system and chemical processes of all tissues, both host and microbes, to survive.

The pH of most drugs act as weak acids or bases that are present in solution as both the non-ionized and ionized species. Non-ionized molecules are usually lipid soluble and can diffuse across the cell membrane. In contrast, the ionized molecules are usually unable to penetrate the lipid membrane because of their low lipid solubility.

Ascorbic acid (AA) is one of the most important water-soluble antioxidants in biological systems. It is believed to be essential for scavenging free radicals, thereby reducing the mutation rate of DNA and preventing the development of diseases associated with gene mutations.

AA plays an integral role in the synthesis of connective tissue and reduces biochemical cofactors and substrates.

To exhibit it microbicidal properties at the host target tissues, its potency must be unaltered in the packaging and delivery method. It has been documented that the inside surface of glassware may contain materials that measurably degrade AA within a short period of time (<24 h). The source of AA degradation may also occur from one or more transition metals that catalyze the oxidation of AA. Such degradation was observed in amber vials and to a lesser extent, with clear autosampler vials. The presence of transition metal cations in the oxidized state bound to the inner surface of the glassware could easily account for the oxidation of AA to dehydroascorbic acid and further degradation products in biological samples.

An exemplary embodiment of the inventive composition disclosed herein includes an essential nutrient for man, L-Ascorbic acid, in the purest physical and natural form, and cardinal low pH. Unfortunately, man is unable synthesize L-Ascorbic acid due to the absence of the biochemical step in the liver of the species to convert L-gulonolactone, which is required for the biosynthesis of L-ascorbic acid. The exemplary embodiment of the composition provides L-Ascorbic acid, (also known as Vitamin C, hexuronic acid, an antisorbutic vitamin) with the chemical formula C₆H₈O₆ and a molecular weight of 176.12, or its derivatives: Ester-C, Ascorbate, L-xyloascorbic acid, 3-oxo-L-gulofuranolactone (enol form), L-3-keto threo hexuronic acid lactone, dehydro ascorbic acid, hydrophobic ascorbic acid, and 2-O-.alpha-D-glocopyranosyl-L-Ascorbic acid, or a suitable derivative in therapeutically effective amounts at the approximate effective pH for a safe and optimal effect in a pharmaceutically acceptable liquid carrier for topical application to the genitalia or anal area to prevent or treat diseases, therein.

This embodiment and the methods described subsequently assure the local availability of the composition directly to the immuno-defense tissues of the genitailia and anorectal area.

When L-ascorbic acid is dissolved in an aqueous solution, the pH becomes sharply acidic due to the dissociation of the hydrogen ion from the enediol group. The main contribution to the lowering of the pH level is the hydroxy group located on the number 3 carbon (C3).

The proposed embodiment employs this essential nutrient, L-Ascorbic Acid in the purest therapeutic form, novel pH, and application as a microbicidal agent and co-enzyme required by man for the production of fibrin and pre-collagen necessary for the healing of impaired tissues. The L-Ascorbic acid or comparable ingredient is in the form of U.S.P. grade, extra pure fine powder, granular powder or pure crystals.

In the pure fine powder form, L-Ascorbic acid remains in solution longer than other physical forms. The isotonic composition generates the osmotic gradient to initiates the absorption of the composition. For the exemplary embodiment, L-Ascorbic acid is employed in a concentration of about 2% to about 25% wt/vol., within the acidic range or pH level of about 4.0 to 2.0 pH respectively. The predetermined low pH of the Embodiment exhibits the most potent specific microbicidal effect and the deformation of biofilm. The diffusible property modifies the pH of the resulting media to approximate the natural pH milieu for the local organ homeostasis.

For a first exemplary embodiment, the liquid carrier for the aqueous solution is normal 0.9% physiologic saline, in a liquid or semi-liquid physical state, in a pre determined composition for the desired therapeutic effect.

Additionally, an antioxidant such as flavone, bioflavonoid, Tocopherol or water solubilized vitamin E (alpha-tocopherol), is added in alternative embodiments to increase the capillary permeability of the composition. Apha-tocopheral is added in alternative embodiments to increase lipid solubility and enhance the antioxidant properties of the composition. A first exemplary embodiment employs a concentration of about 0.1 to 0.5% wt/vol of flavone. A second exemplary embodiment employs a concentration of about 500 to 1,000 i.u./100 m vitamin E.

An essential metalo-enzyme, the trace element, zinc, is added in certain embodiments to facilitate the biochemical recovery of the immune tissues.

The method of delivery, of the semi liquid composition, as described herein, and employed is most consistent with the desired therapeutic effect of the novel composition to prevent the transmission of infection and restore local tissue health the immune defenses of the genital membranes.

The preparation process of the composition is designed to maintain the maximum biochemical therapeutic effect described herein and method to minimize the Maillard reaction by controlling the parameters of the Composition; its isotonicity, osmolar activity, critical pH range, temperature, antioxidant and surfactant chemical properties. The composition may be modified for the synergy of the homeorrhesis of the host organ and tissue immune defenses against encroaching microbes.

Application of the composition is accomplished topically in several physical forms according to the present invention, described herein. For the embodiment delivered to the female genitalia; For the prophylaxis of conception, vaginosis and STDs, including Human Immunodeficiency Virus (HIV) infections, the liquid carrier comprises physiologic saline in a pre determined composition for the therapeutic effect and topical application is provided by a rinse or douche delivered into the vagina. The aqueous solution is employed in an alternative embodiment to saturate a tampon which then provides delivery of the solution into the vagina during use.

Alternatively, the composition is provided in the form of a cream, gel or suppository which is delivered into the genitalia or anorectal area of the receptive partner. When employed as a cream or gel, the preparation of the composition is formulated in an oil and water-based emulsion and includes emollients that change the rheology of the composition to the semi liquid state, while synergistically potentiating the microbicidal impact in the local milieu. This may include propylene glycol, glycerin, mineral oil, Cetyl emollients and xantham gum. The vaginal inserts can comprise a mixture or combination of water soluble pore forming agents; polyethylene glycol (PEG), that provide release of the active agent over a prolonged time interval at a constant rate. The vaginal inserts can comprise a mixture or combination with water insoluble Polymers to extend the release time of the embodiment described herein.

An alternative embodiment incorporates the saturation of the external surface of a condom with the solution, cream or gel for delivery of the composition during intercourse.

In exemplary embodiments for use on the female genitalia, efficacious solutions have been demonstrated with a pH in the range of about 2.2 pH to about 3.0 pH level or in which the L-Ascorbic acid is present in the carrier at a concentration from about 2% to about 10% wt./volume.

For application to the male genitalia, to prevent and treat infection (ballanitis) and for the prevention of the Human Immunodeficiency Virus (HIV) infection, the previously described composition is delivered onto the membranes of the glans of the penis in the form of drops, a spray, or a cleansing rinse. Similarly, the composition is delivered onto the membranes of the membranes of the prepuce, foreskin, in the form of drops, a cleansing spray or a cleansing rinse.

Alternatively, the composition is delivered onto the membranes of the glans or foreskin of the penis in the form of a cleansing cream or a cleansing gel as previously described with respect to vaginal treatment.

Additionally, in an alternative embodiment, the composition is delivered onto the penis in the form of a condom saturated on its interior with the previously described aqueous solution or the modified cream or gel.

In exemplary embodiments of the composition for use on the male genitalia, efficacious solutions have been demonstrated with a pH level of 2.2 pH to about 2.6 pH or in which the L-Ascorbic acid is present in the carrier at a concentration from about 2% to about 10% wt./volume.

Similarly, delivery to the anorectal area is accomplished in the form of a cleansing rinse, a cleansing cream or a cleansing gel. Alternatively, the composition is delivered onto the rectum through use of a condom saturated externally with the previously described aqueous solution or the modified cream or gel as previously described with respect to vaginal treatment.

Alternatively, the composition is delivered onto the membranes of the rectum in the form of a suppository as previously described with respect to a vaginal suppository form.

In exemplary embodiments of the composition for use on the rectum, efficacious solutions have been demonstrated with a pH level of 2.2 pH to about 2.9 pH or in which the L-Ascorbic acid is present in the carrier at a concentration from about 2% to about 10% wt./volume.

### Selected Case Studies and Exemplary Treatment Issues

A 64 year-old female with a history of surgical menopause following a total hysterectomy a number of years ago has suffered from very dry vaginal membranes, dyspareunia and occasional vaginitis. She declined the administration of estrogen or female hormones because of the reported incidence of increased cancer of the breast, and her own positive family history of the same.

She used the special embodiment described herein as a vaginal weekly as needed to maintained vaginal hygiene, its microbicidal protection and the lubrication of the vaginal membranes. She has been free of vaginal infections since undertaking the program.

The composition and method of the present invention preserves the pH of the acidic mantel for normal primary vaginal defenses, capable of inhibiting the HIV at the initial point of contact with surfactant-like properties to deform the biofilm shield and with antioxidant co-enzymes factors that support the production of pre-collagen for host tissue strength. But it must be universally safe, both for teenagers and the post-menopausal age group, for the delicate host tissues. Such is the Method and Composition of the novel Embodiment described herein.

### Genitalia

Selected Case Studies:

Male Infections

A 40 year-old male with a past history of a Chlamydia infection and a concern about being at risk for other STD's diseases if exposed to future partners, was counseled. This uncircumcised male used the embodiment as described herein. The embodiment was applied to the inner aspect of the foreskin and over the glands of the penis including the urethral orifice. The embodiment was applied both before, and after, coitus. Whether or not the embodiment perfused condom was used during intercourse, the embodiment was applied to the genital area, before and after, in the manner described herein. He has had no subsequent genital infections related to STD's.

This 46 year-old receptive partner in ano-genital intercourse was HIV positive, but not clinically active. For prophylaxis he delivered the embodiment described above into the ano-rectal area before and after ano-rectal sex activities. His partner applied the embodiment described herein to coat the inner surface of the foreskin and the glands. When a condom was used the prophylactic was profused with the embodiment described herein. There has been no transmission of the HIV to the dominant partner nor activation of the pre-existing HIV in the receptive partner.

A principal target of the present Invention is to reduce the initial male-to-female HIV transmission of the active HIV while it remains within the vaginal cavity. The microbicide properties of the composition described herein used topically disrupts the biofilm layer and inhibits the organism upon contact while safely and synergistically restoring the natural immune defenses and healing processes of the genital membranes.

By lowering the pH of the vaginal environment, the rheologic properties of the protective biofilm covering the micro-organism are deformed, the microbial virulence is impaired at the point of contact, and penetration of the host defenses is interrupted. If the HIV cannot reach the Langerhan receptor cells, critical attachment will not occur. In the same biochemical mechanism, spermatozoa motility is significantly inhibit sperm and viability.

The method and composition described herein is intended to inactivate the HIV directly upon contact, and indirectly by supporting the natural immune defenses in the vagina counteract penetration by the virus or microbe while it is within the cavity of the vagina. Additionally, this novel invention has been developed specifically to alter bacterial adherence while enabling the vagina in maintaining the critical low pH for homeostasis and tissue immune defense, and to counteract the alkaline pH of seminal fluids in the vagina.

Having now described the invention in detail as required by the patent statutes, those skilled in the art will recognize modifications and substitutions to the specific embodiments disclosed herein. Such modifications are within the scope and intent of the present invention as defined in the following claims.

What is claimed is:

1. A method for treatment of symptoms of inflammation and dyspareunia with respect to vaginoses, vaginitis and HIV comprising topical application to the vagina of L-Ascorbic acid in a concentration of about 2% to about 25% wt/vol., within an acidic range or pH level of about 4.0 to 2.0 pH respectively, in an aqueous solution with a pharmaceutically acceptable liquid carrier.

2. The method of claim 1 wherein topical application is accomplished in a form selected from the set of a douche, rinse, cream, gel, suppository, saturated tampon and externally saturated condom.

3. The method of claim 1 wherein the pH level is between 2.2 pH to about 3.0 pH.

4. A method for treatment of symptoms of inflammation and balanitis with respect to chlamydia and HIV comprising topical application to the glans and foreskin of a penis of L-Ascorbic acid in a concentration of about 2% to about 25% wt/vol., within an acidic range or pH level of about 4.0 to 2.0 pH respectively, in an aqueous solution with a pharmaceutically acceptable liquid carrier.

5. The method of claim 4 wherein topical application is accomplished in a form selected from the set of drops, spray, cleansing wipe, cream, gel and internally saturated condom.

6. The method of claim 4 wherein the pH level is between 2.2 pH to about 2.6 pH.

7. A method for treatment of symptoms of inflammation with respect to chlamydia and HIV comprising topical application to the rectum of L-Ascorbic acid in a concentration of about 2% to about 25% wt/vol., within an acidic range or pH level of about 4.0 to 2.0 pH respectively, in an aqueous solution with a pharmaceutically acceptable liquid carrier.

8. The method of claim 7 wherein topical application is accomplished in a form selected from the set of cleansing wipe, gel, suppository and externally saturated condom.

9. The method of claim 7 wherein the pH level is between 2.2 pH to about 2.9 pH.
